SEQUENCE LISTING (22 sequences)

SEQ ID NO: 1 - protein, 5 aa, synthetic construct
  REGION 1..5: note = SEQ ID NO.: 1 encodes for a CDR (VHCDR1) of AbD33905.
SYAMH

SEQ ID NO: 2 - protein, 17 aa, synthetic construct
  REGION 1..17: note = SEQ ID NO.: 2 encodes for a CDR (VHCDR2) of AbD33905.
FISSGGSTTYYADSVKG

SEQ ID NO: 3 - protein, 8 aa, synthetic construct
  REGION 1..8: note = SEQ ID NO.: 3 encodes for a CDR (VHCDR3) of AbD33905.
FRSGYFDY

SEQ ID NO: 4 - protein, 16 aa, synthetic construct
  REGION 1..16: note = SEQ ID NO.: 4 encodes for a CDR (VLCDR1) of AbD33905.
RSSQSLVHSNGADYLN

SEQ ID NO: 5 - protein, 7 aa, synthetic construct
  REGION 1..7: note = SEQ ID NO.: 5 encodes for a CDR (VLCDR2) of AbD33905.
KGSNRDS

SEQ ID NO: 6 - protein, 10 aa, synthetic construct
  REGION 1..10: note = SEQ ID NO.: 6 encodes for a CDR (VLCDR3) of AbD33905.
QQGYDFPPFT

SEQ ID NO: 7 - protein, 5 aa, synthetic construct
  REGION 1..5: note = SEQ ID NO.: 7 encodes for a CDR (VHCDR1) of AbD33906.
SYAIS

SEQ ID NO: 8 - protein, 17 aa, synthetic construct
  REGION 1..17: note = SEQ ID NO.: 8 encodes for a CDR (VHCDR2) of AbD33906.
GIIPQFGIANYAQKFQG

SEQ ID NO: 9 - protein, 11 aa, synthetic construct
  REGION 1..11: note = SEQ ID NO.: 9 encodes for a CDR (VHCDR3) of AbD33906.
EGYSDYSAFDY

SEQ ID NO: 10 - protein, 14 aa, synthetic construct
  REGION 1..14: note = SEQ ID NO.: 10 encodes for a CDR (VLCDR1) of AbD33906.
TGTSSDVGTYNYVN

SEQ ID NO: 11 - protein, 7 aa, synthetic construct
  REGION 1..7: note = SEQ ID NO.: 11 encodes for a CDR (VLCDR2) of AbD33906.
RVSNRPS

SEQ ID NO: 12 - protein, 9 aa, synthetic construct
  REGION 1..9: note = SEQ ID NO.: 12 encodes for a CDR (VLCDR3) of AbD33906.
SSWDEHSYV

SEQ ID NO: 13 - protein, 117 aa, synthetic construct
  REGION 1..117: note = SEQ ID NO.: 13 encodes the VH domain of AbD33905.
EVQLLESGGGLVQPGGSLRLSCAASGFTFSSYAMHWVRQAPGKGLEWVSFISSGGSTTYYADSVKGRFTISRDNSKNTLYLQMNSLRAEDTAVYYCARFRSGYFDYWGQGTLVTVSS

SEQ ID NO: 14 - protein, 115 aa, synthetic construct
  REGION 1..115: note = SEQ ID NO.: 14 encodes the VL domain of AbD33905.
DIVMTQSPLSLPVTPGEPASISCRSSQSLVHSNGADYLNWYLQKPGQSPQLLIYKGSNRDSGVPDRFSGSGSGTDFTLKISRVEAEDVGVYYCQQGYDFPPFTFGQGTKVEIKRT

SEQ ID NO: 15 - protein, 120 aa, synthetic construct
  REGION 1..120: note = SEQ ID NO.: 15 encodes the VH domain of AbD33906.
QVQLVQSGAEVKKPGSSVKVSCKASGGTFSSYAISWVRQAPGQGLEWMGGIIPQFGIANYAQKFQGRVTITADESTSTAYMELSSLRSEDTAVYYCAREGYSDYSAFDYWGQGTLVTVSS

SEQ ID NO: 16 - protein, 111 aa, synthetic construct
  REGION 1..111: note = SEQ ID NO.: 16 encodes the VL domain of AbD33906.
DIALTQPASVSGSPGQSITISCTGTSSDVGTYNYVNWYQQHPGKAPKVMIYRVSNRPSGVSNRFSGSKSGNTASLTISGLQAEDEADYYCSSWDEHSYVFGGGTKLTVLGQ

SEQ ID NO: 17 - protein, 219 aa, synthetic construct
  REGION 1..219: note = SEQ ID NO.: 17 encodes the VH and CH1 domain of AbD33905.
EVQLLESGGGLVQPGGSLRLSCAASGFTFSSYAMHWVRQAPGKGLEWVSFISSGGSTTYYADSVKGRFTISRDNSKNTLYLQMNSLRAEDTAVYYCARFRSGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 18 - protein, 220 aa, synthetic construct
  REGION 1..220: note = SEQ ID NO.: 18 encodes the VL and CL domain of AbD33905.
DIVMTQSPLSLPVTPGEPASISCRSSQSLVHSNGADYLNWYLQKPGQSPQLLIYKGSNRDSGVPDRFSGSGSGTDFTLKISRVEAEDVGVYYCQQGYDFPPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 19 - protein, 222 aa, synthetic construct
  REGION 1..222: note = SEQ ID NO.: 19 encodes the VH and CH1 domain of AbD33906.
QVQLVQSGAEVKKPGSSVKVSCKASGGTFSSYAISWVRQAPGQGLEWMGGIIPQFGIANYAQKFQGRVTITADESTSTAYMELSSLRSEDTAVYYCAREGYSDYSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 20 - protein, 214 aa, synthetic construct
  REGION 1..214: note = SEQ ID NO.: 20 encodes the VL and CL domain of AbD33906.
DIALTQPASVSGSPGQSITISCTGTSSDVGTYNYVNWYQQHPGKAPKVMIYRVSNRPSGVSNRFSGSKSGNTASLTISGLQAEDEADYYCSSWDEHSYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 21 - protein, 723 aa, Homo sapiens
  REGION 1..723: note = SEQ ID NO.: 21 encodes a DLL1 isoform (UniProtKB O00548(DLL1_HUMAN).
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 22 - protein, 247 aa, Homo sapiens
  REGION 1..247: note = SEQ ID NO.: 22 encodes another DLL1 isoform (UniProtKB O00548-2(DLL1_HUMAN).
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTERESLGRHRWLTRPRTRTTRRDGAS

(19) United States
(12) Patent Application Publication
(10) Pub. No.: US 2025/0052768 A1
(43) Pub. Date: Feb. 13, 2025

(54) IN VITRO METHODS FOR DETECTING DLL1

(71) Applicant: Universität Heidelberg, Heidelberg (DE)

(72) Inventors: Klaus Heeg, Heidelberg (DE); Markus A. Weigand, Wettenberg (DE); Dagmar Hildebrand, Dossenheim (DE); Florian Uhle, Heidelberg (DE)

(21) Appl. No.: 18/695,282

(22) PCT Filed: Sep. 28, 2022
(86) PCT No.: PCT/EP2022/076950

§ 371 Date: Mar. 25, 2024

(30) Foreign Application Priority Data
Sep. 28, 2021 (EP) 21199512.1

(51) Int. Cl. G01N 33/68; G01N 33/543; G01N 33/58

(52) U.S. Cl. CPC G01N 33/6893; G01N 33/54388; G01N 33/58; G01N 2333/705; G01N 2800/26

(57) ABSTRACT

The invention relates to in vitro methods for detecting Delta-like 1 (DLL1) protein using an anti-DLL 1 capture antibody and a labeled anti-DLL1 detection antibody. According to the invention, these antibodies can be used in an in vitro method to diagnose a severe infection, in particular a sepsis.

### FIELD OF THE INVENTION

The invention relates to in vitro methods for detecting Delta-like 1 (DLL1) protein qualitatively and quantitatively using an anti-DLL1 capture antibody and/or a labeled anti-DLL1 detection antibody. In this context, the invention also relates to the use of the anti-DLL1 capture antibody and/or the labeled anti-DLL1 detection antibody in an in vitro method to diagnose a severe infection, in particular a sepsis.

### BACKGROUND OF THE INVENTION

Severe infections including sepsis are identified by and/or may result in life-threatening organ failure evoked by a dysregulated immune response to infection. In sepsis, the host response that is triggered by microbial pathogens peaks in a pathological syndrome. This syndrome is characterized by exaggerated inflammation and a subsequent immune suppression.

Despite the steady improvements in critical care medicine and anti-microbial therapies, such infection remains a leading cause of death in intensive care units across all age groups worldwide. Early diagnosis is necessary to properly manage these severe infections as the initiation of rapid therapy is key to reducing deaths from them. Thus, a rapid and reliable method for diagnosing a severe infection, which ideally can be performed near or at the point of patient care, is needed.

Up to now, mainly blood cultures are used as a gold standard in diagnosing a sepsis. Such blood cultures, however, take time and cannot be performed near or at the point of patient care. Moreover, many patients, who show signs and symptoms of sepsis, have negative blood culture results.

Recently, Delta-like protein 1 (DLL1) was identified as suitable biomarker for the in vitro diagnosis of a severe infection (WO2019/081636 and Hildebrand, Dagmar, et al. “Host-Derived Delta-Like Canonical Notch Ligand 1 as a Novel Diagnostic Biomarker for Bacterial Sepsis—Results From a Combinational Secondary Analysis.” Frontiers in cellular and infection microbiology 9 (2019): 267).

Delta-like proteins are single-pass transmembrane proteins known for their role in Notch signaling. Synonyms of DLL1 are delta-like-ligand 1, delta-like protein, H-Delta, drosophila Delta homolog 1, delta like canonical Notch ligand 1, DL1, and Notch ligand deltal-like1. In mammals, there are three delta-like genes encoding delta-like ligand 1 (dll1 encoding DLL1), delta-like ligand 3 (dll3 encoding DLL3), and delta-like ligand 4 (dll4 encoding DLL4). All delta-like ligands comprise a conserved cysteine-rich region known as the DSL (Delta, Serrate, Lag2) domain, several epidermal growth factor (EGF)-like repeats, and a transmembrane domain. The amino acid sequence of delta-like ligand 1 protein and the nucleotide sequence coding for delta-like ligand 1 protein are known. For example, an amino acid sequence of DLL1 is described in American Journal of Pathology, Vol. 154, No.3, March 1999, 785-794 or in the database of the National Center for Biotechnology Information (https://www.ncbi.nlm.nih.gov/protein/NP_005609.3).

Clinically suitable methods, let alone point-of-care tests, which make use of this new DLL1 biomarker for diagnosing a severe infection including a sepsis are, however, missing.

### SUMMARY OF THE INVENTION

Against the aforedescribed background, it is an object of the present invention to provide a (clinical) method to rapidly and reliably diagnose a severe infection including a sepsis. In particular, it is an object of the present invention to provide a rapid and reliable (clinical) method to diagnose a severe infection such as a sepsis which makes use of DLL1 as a biomarker. It is a further object of the invention to provide such a method in form of a point-of-care test, which can be performed quickly and reliably at the time and place of patient care.

These objects are achieved by the in vitro method according to claim 1 and the use according to claim 10.

The invention provides an in vitro method for detecting DLL1 protein qualitatively and quantitatively using
a. a labeled anti-DLL1 detection antibody, or
b. an anti-DLL1 capture antibody, or
c. a labeled anti-DLL1 detection antibody and an anti-DLL1 capture antibody wherein the labeled anti-DLL1 detection antibody and/or the anti-DLL1 capture antibody are selected from the group consisting of:
i) an isolated antibody or antigen-binding portion thereof comprising at least one complementary determining region (CDR) selected from SEQ ID NOs.: 1-6; and
ii) an isolated antibody or antigen-binding portion thereof comprising at least one CDR selected from SEQ ID NOs.: 7-12.

The labeled anti-DLL1 detection antibody or the anti-DLL1 capture antibody of the invention work surprisingly well to specifically and/or preferentially bind and detect DLL1 protein at low levels, in particular in human samples such as blood samples, plasma samples or serum samples. Further, the research underlying the invention surprisingly showed that the anti-DLL1 capture antibody and the labeled anti-DLL1 detection antibody according to the invention work particularly well in combination with each other to specifically and/or preferentially bind and detect DLL1 protein at low levels. The detection of DLL1 protein is even more efficient in the method of the invention when the anti-DLL1 capture antibody and the labeled anti-DLL1 detection antibody do not belong to the same alternative i) or ii). Particularly good results are achieved, when the antibody according to alternative i) is used as a labeled anti-DLL1 detection antibody and the antibody according to alternative ii) as anti-DLL1 capture antibody. For this case, studies of the inventors have shown that particularly low levels of DLL1 protein can be still detected. This was most surprising.

Lastly, the invention concerns the use of a labeled anti-DLL1 detection antibody and/or of an anti-DLL1 capture antibody according to the invention in an in vitro method to diagnose a severe infection, in particular a sepsis. Experiments of the inventors have shown that these antibodies are particularly suitable to specifically detect DLL1 protein at low levels in a biological sample such as a plasma or serum sample.

### DETAILED DESCRIPTION OF ILLUSTRATIVE EMBODIMENTS

### The In Vitro Method

The in vitro method according to the invention allows to detect DLL1 protein qualitatively and quantitatively. To this end, the in vitro method of the invention employs a labeled anti-DLL1 detection antibody and/or an anti-DLL1 capture antibody selected from the group consisting of:
i) an isolated antibody or antigen-binding portion thereof comprising at least one complementary determining region (CDR) amino acid sequence selected from SEQ ID NOs.: 1-6; and
ii) an isolated antibody or antigen-binding portion thereof comprising at least one CDR amino acid sequence selected from SEQ ID NOs.: 7-12.

Such anti-DLL1 detection antibodies or anti-DLL1 capture antibodies of the invention work surprisingly well to specifically and/or preferentially bind and detect DLL1 protein at low levels, in particular in human samples such as blood samples, plasma samples or serum samples.

It is to be noted that the term “a” or “an” entity refers to one or more of that entity, e.g., “a labeled anti-DLL1 detection antibody” or “an anti-DLL1 capture antibody” is understood to represent one or more antibodies of this kind.

In certain embodiments, the isolated antibody or antigen-binding portion thereof according to alternative i) comprises a variable domain of a heavy chain (VH) and a variable domain of a light chain (VL), wherein the VH comprises a CDR amino acid sequence (VHCDR) selected from SEQ ID NOs: 1-3.

In certain embodiments, the isolated antibody or antigen-binding portion thereof according to alternative i) comprises a VH domain and a VL domain, wherein the VH comprises a VHCDR1 amino acid sequence of SEQ ID NO.: 1.

In certain embodiments, the isolated antibody or antigen-binding portion thereof according to alternative i) comprises a VH domain and VL domain, wherein the VH comprises a VHCDR2 amino acid sequence of SEQ ID NO.: 2.

In certain embodiments, the isolated antibody or antigen-binding portion thereof according to alternative i) comprises a VH domain and a VL domain, wherein the VH comprises a VHCDR3 amino acid sequence of SEQ ID NO.: 3.

In certain embodiments, the isolated antibody or antigen-binding portion thereof according to alternative i) comprises a VH domain and a VL domain, wherein the VH comprises a VHCDR1 of SEQ ID NO.: 1 and a VHCDR2 of SEQ ID NO: 2; or a VHCDR1 of SEQ ID NO.: 1 and a VHCDR3 of SEQ ID NO: 3; or a VHCDR2 of SEQ ID NO: 2 and a VHCDR3 of SEQ ID NO: 3.

In certain embodiments, the isolated antibody or antigen-binding portion thereof according to alternative i) comprises a VH domain and a VL domain, wherein the VH comprises VHCDR1, VHCDR2, and VHCDR3 amino acid sequences of SEQ ID NOs: 1, 2, and 3, respectively.

In certain embodiments, the isolated antibody or antigen-binding portion thereof according to alternative i) comprises a VH domain and a VL domain, wherein the VL comprises a CDR amino acid sequence (VLCDR) selected from SEQ ID NOs: 4-6.

In certain embodiments, the isolated antibody or antigen-binding portion thereof according to alternative i) comprises VH domain and a VL domain, wherein the VL comprises a VLCDR1 amino acid sequence of SEQ ID NO.: 4.

In certain embodiments, the isolated antibody or antigen-binding portion thereof according to alternative i) comprises VH domain and a VL domain, wherein the VL comprises a VLCDR2 amino acid sequence of SEQ ID NO.: 5.

In certain embodiments, the isolated antibody or antigen-binding portion thereof according to alternative i) comprises VH domain and a VL domain, wherein the VL comprises a VLCDR3 amino acid sequence of SEQ ID NO.: 6.

In certain embodiments, the isolated antibody or antigen-binding portion thereof according to alternative i) comprises a VH domain and a VL domain, wherein the VL comprises a VLCDR1 of SEQ ID NO.: 4 and a VLCDR2 of SEQ ID NO: 5; or a VLCDR1 of SEQ ID NO.: 4 and a VLCDR3 of SEQ ID NO: 6; or a VLCDR2 of SEQ ID NO: 5 and a VLCDR3 of SEQ ID NO: 6.

In certain embodiments, the isolated antibody or antigen-binding portion thereof according to alternative i) comprises a VH domain and a VL domain, wherein the VL comprises VLCDR1, VLCDR2, and VLCDR3 amino acid sequences of SEQ ID NOs: 4, 5, and 6, respectively.

In certain embodiments, the isolated antibody or antigen-binding portion thereof according to alternative ii) comprises a VH domain and a VL domain, wherein the VH comprises a CDR amino acid sequence selected from SEQ ID NOs: 7-9.

In certain embodiments, the isolated antibody or antigen-binding portion thereof according to alternative ii) comprises a VH domain and a VL domain, wherein the VH comprises a VHCDR1 amino acid sequence of SEQ ID NO.: 7.

In certain embodiments, the isolated antibody or antigen-binding portion thereof according to alternative ii) comprises a VH domain and VL domain, wherein the VH comprises a VHCDR2 amino acid sequence of SEQ ID NO.: 8.

In certain embodiments, the isolated antibody or antigen-binding portion thereof according to alternative ii) comprises a VH domain and a VL domain, wherein the VH comprises a VHCDR3 amino acid sequence of SEQ ID NO.: 9.

In certain embodiments, the isolated antibody or antigen-binding portion thereof according to alternative ii) comprises a VH domain and a VL domain, wherein the VH comprises a VHCDR1 of SEQ ID NO.: 7 and a VHCDR2 of SEQ ID NO: 8; or a VHCDR1 of SEQ ID NO.: 7 and a VHCDR3 of SEQ ID NO: 9; or a VHCDR2 of SEQ ID NO: 8 and a VHCDR3 of SEQ ID NO: 9.

In certain embodiments, the isolated antibody or antigen-binding portion thereof according to alternative ii) comprises a VH domain and a VL domain, wherein the VH comprises VHCDR1, VHCDR2, and VHCDR3 amino acid sequences of SEQ ID NOs: 7, 8, and 9, respectively.

In certain embodiments, the isolated antibody or antigen-binding portion thereof according to alternative ii) comprises a VH domain and a VL domain, wherein the VL comprises a CDR amino acid sequence selected from SEQ ID NOs: 10-12.

In certain embodiments, the isolated antibody or antigen-binding portion thereof according to alternative ii) comprises VH domain and a VL domain, wherein the VL comprises a VLCDR1 amino acid sequence of SEQ ID NO.: 10.

In certain embodiments, the isolated antibody or antigen-binding portion thereof according to alternative ii) comprises VH domain and a VL domain, wherein the VL comprises a VLCDR2 amino acid sequence of SEQ ID NO.: 11.

In certain embodiments, the isolated antibody or antigen-binding portion thereof according to alternative ii) comprises VH domain and a VL domain, wherein the VL comprises a VLCDR3 amino acid sequence of SEQ ID NO.: 12.

In certain embodiments, the isolated antibody or antigen-binding portion thereof according to alternative ii) comprises a VH domain and a VL domain, wherein the VL comprises a VLCDR1 of SEQ ID NO.: 10 and a VLCDR2 of SEQ ID NO: 11; or a VLCDR1 of SEQ ID NO.: 10 and a VLCDR3 of SEQ ID NO: 12; or a VLCDR2 of SEQ ID NO: 11 and a VLCDR3 of SEQ ID NO: 12.

In certain embodiments, the isolated antibody or antigen-binding portion thereof according to alternative ii) comprises a VH domain and a VL domain, wherein the VL comprises VLCDR1, VLCDR2, and VLCDR3 amino acid sequences of SEQ ID NOs: 10, 11, and 12, respectively.

Anti-DLL1 detection antibodies or anti-DLL1 capture antibodies according to the aforedescribed embodiments work particularly well to specifically and/or preferentially bind and detect DLL1 protein at low levels.

In a further embodiment of the invention, the labeled anti-DLL1 detection antibody and/or the anti-DLL1 capture antibody are selected from the group consisting of:
i) an isolated antibody or antigen-binding portion thereof comprising SEQ ID NO.: 1-6 as CDR amino acid sequences; and
ii) an isolated antibody or antigen-binding portion thereof comprising SEQ ID NO.: 7-12 as CDR amino acid sequences.

According to this embodiment, the antibody or antigen-binding portion thereof comprises as CDR amino acid sequences of the VH and VL domain, SEQ ID NO.: 1, 2, 3, 4, 5 and 6 according to alternative i) and SEQ ID NO.: 7, 8, 9, 10, 11, and 12 according to alternative ii).

In certain embodiments, the isolated antibody or antigen-binding portion thereof according to alternative i) comprises a VH domain and a VL domain, wherein the VH comprises VHCDR1, VHCDR2, and VHCDR3 amino acid sequences of SEQ ID NOs: 1, 2, 3, and the VL comprises VLCDR1, VLCDR2, VLCDR3 amino acid sequences of SEQ ID NOs: 4, 5, 6 and the isolated antibody or antigen-binding portion thereof according to alternative ii) comprises a VH domain and a VL domain, wherein the VH comprises VHCDR1, VHCDR2, and VHCDR3 amino acid sequences of SEQ ID NOs: 7, 8, 9, and the VL comprises VLCDR1, VLCDR2, VLCDR3 amino acid sequences of SEQ ID NOs: 10, 11, 12. Experiments of the inventors have shown that such antibodies or antigen-binding portions thereof are particularly effective to specifically and/or preferentially bind and detect DLL1 protein at low levels.

The labeled anti-DLL1 detection antibody and the anti-DLL1 capture antibody according to the invention bind specifically and/or preferentially to DLL1 protein.

By “specifically binds”, it is generally meant that an antibody or the antigen-binding portion thereof binds to an epitope via its antigen binding domain, and that the binding is based on some complementarity between the antigen binding domain and the epitope on the antigen. According to this definition, an antibody is said to “specifically bind” to an epitope when it binds to that epitope via its antigen binding domain more readily than it would bind to a random, unrelated epitope. The term “specificity” is used herein to qualify the relative affinity by which a certain antibody binds to a certain epitope. For example, antibody “A” can be deemed to have a higher specificity for a given epitope than antibody “B”, or antibody “A” can be said to bind to epitope “C” with a higher specificity than it has for related epitope “D”.

By “preferentially binds”, it is meant that the antibody or the antigen-binding portion thereof specifically binds to an epitope more readily than it would bind to a related, similar, homologous, or analogous epitope. Thus, an antibody or antigen-binding porition thereof that “preferentially binds” to a given epitope would more likely bind to that epitope than to a related epitope, even though such an antibody can cross-react with the related epitope. For example, an antibody can be considered to bind a first epitope preferentially if it binds said first epitope with a dissociation constant (K_{D}) that is less than the antibody's K_{D} for the second epitope.

The labeled anti-DLL1 detection antibody and the anti-DLL1 capture antibody according to the invention do not substantially bind to DLL3 and DLL4 isoforms. Preferably, the antibodies of the invention do not bind to isoforms of DLL3 and DLL4. This has the advantage that any off-target binding to the related DLL3 and DLL4 isoforms (sequence similiarties to SEQ ID NO: 21 of about 37 to 63% as obtained by the Pairwise Sequence Alignment Tool “EMBOSS Needle” of the EMBL-EBI Hinxton (https://www.ebi.ac.uk/Tools/psa/emboss_needle/) does not take place.

In another embodiment of the invention, the labeled anti-DLL1 detection antibody and/or the anti-DLL1 capture antibody are selected from the group consisting of
i) an isolated antibody or antigen-binding portion thereof comprising
   a heavy chain variable domain (V_{H}) that has at least 95%, preferably at least 98% sequence identity to SEQ ID NO.: 13, and
   a light chain variable domain (V_{L}) that has at least 95%, preferably at least 98% sequence identity to SEQ ID NO.: 14; and
ii) an isolated antibody or antigen-binding portion thereof comprising
   a heavy chain variable domain (V_{H}) that has at least 95%, preferably at least 98% sequence identity to SEQ ID NO.: 15, and
   a light chain variable domain (V_{L}) that has at least 95%, preferably at least 98% sequence identity to SEQ ID NO.: 16.

The research underlying the invention has shown that such antibodies are particularly effective in specifically binding and/or preferentially binding and detecting DLL1 protein at low levels. In this embodiment, the amino acid sequence differences are preferably not present in VHCDR1 (SEQ ID NO.: 1), VHCDR2 (SEQ ID NO.: 2), VHCDR3 (SEQ ID NO.: 3), VLCDR1 (SEQ ID NO.: 4), VLCDR2 (SEQ ID NO.: 5), VLDCDR3 (SEQ ID NO.: 6) in case of alternative i) and in VHCDR1 (SEQ ID NO.: 7), VHCDR2 (SEQ ID NO.: 8), VHCDR3 (SEQ ID NO.: 9), VLCDR1 (SEQ ID NO.: 10), VLCDR2 (SEQ ID NO.: 11), VLDCDR3 (SEQ ID NO.: 12) in case of alternative ii).

In a further embodiment of the invention, the labeled anti-DLL1 detection antibody and/or the anti-DLL1 capture antibody are selected from the group consisting of
i) an isolated antibody or antigen-binding portion thereof comprising
   a V_{H} and CH1 containing sequence that has at least 95%, preferably at least 98% sequence identity to SEQ ID NO.: 17, and
   V_{L} and C_{L} containing sequence that has at least 95%, preferably at least 98% sequence identity to SEQ ID NO.: 18; and
ii) an isolated antibody or antigen-binding portion thereof comprising
   a V_{H} and CH1 containing sequence that has at least 90% sequence identity to SEQ ID NO.: 19; and
   V_{L} and C_{L} containing sequence that has at least 90% sequence identity to SEQ ID NO.: 20.

Experiments of the inventors have shown that such antibodies are particularly effective in specifically binding and detecting DLL1 protein at low levels. In this embodiment, the amino acid sequence differences are preferably not present in VHCDR1 (SEQ ID NO.: 1), VHCDR2 (SEQ ID NO.: 2), VHCDR3 (SEQ ID NO.: 3), VLCDR1 (SEQ ID NO.: 4), VLCDR2 (SEQ ID NO.: 5), VLDCDR3 (SEQ ID NO.: 6) in case of alternative i) and in VHCDR1 (SEQ ID NO.: 7), VHCDR2 (SEQ ID NO.: 8), VHCDR3 (SEQ ID NO.: 9), VLCDR1 (SEQ ID NO.: 10), VLCDR2 (SEQ ID NO.: 11), VLDCDR3 (SEQ ID NO.: 12) in case of alternative ii).

The terms “antibody” and “immunoglobulin” are used interchangeably herein. An antibody or immunoglobulin comprises at least the constant domain of a heavy chain, and normally comprises at least the variable domains of a heavy chain and a light chain.

As used herein, the term “antibody” comprises various broad classes of polypeptides that can be distinguished biochemically. The heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD, or IgE, respectively. The immunoglobulin subclasses (isotypes), e.g., IgG1, IgG2, IgG3, IgG4, are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration. Light chains are classified as either kappa or lambda (κ, λ). Each heavy chain class can be bound with either a kappa or lambda light chain.

Both the light and heavy chains are divided into regions of structural and functional homology. In this regard, the variable domains of both the light (VL) and heavy (VH) chain portions determine antigen recognition and specificity. In contrast, the constant domains of the light chain (CL) and the heavy chain (CH1, CH2 or CH3) confer important biological properties such as secretion, Fc receptor binding and complement binding. By convention the numbering of the constant region domains increases as they become more distal from the antigen binding site or amino-terminus of the antibody.

The variable region allows the antibody to selectively recognize and specifically bind epitopes on antigens. In this regard, the VL domain and VH domain, specifically the subset of the complementarity determining regions (CDRs) within these variable domains, combine to form the variable region that defines a three dimensional antigen binding site. This quaternary antibody structure forms the antigen binding site present at the end of each arm of the Y. More specifically, the antigen binding site is defined by three CDRs on each of the VH and VL chains.

In naturally occurring antibodies, the six CDRs present in each antigen binding domain are short, non-contiguous sequences of amino acids that are specifically positioned to form the antigen binding domain as the antibody assumes its three dimensional configuration. The remainder of the amino acids in the antigen binding domains, referred to as “framework” regions, show less inter-molecular variability. The framework regions largely adopt a β-sheet conformation and the CDRs form loops that connect, and in some cases form part of the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on antigen. This complementary surface promotes the non-covalent binding of the antibody to its cognate epitope. The amino acids comprising the CDRs and the framework regions, respectively, can be readily identified for any given heavy or light chain variable domain by the skilled person, since they have been precisely defined by Kabat et al. (1983) U.S. Dept. of Health and Human Services, “Sequences of Proteins of Immunological Interest” and Chothia and Lesk, J Mol. Biol. 796:901-917 (1987).

The appropriate amino acid residues that encompass the CDRs as defined by each of the above cited references are set forth below in Table 1 as a comparison. The exact residue numbers that encompass a particular CDR will vary depending on the sequence and size of the CDR.

TABLE 1
CDR Definitions. The numbering of all CDR definitions in Table 1 is
according to the numbering conventions set forth by Kabat et al.

| | Kabat et al. | Chothia and Lesk |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

The numbering system for variable domain sequences according to Kabat et al. is applicable to any antibody. The skilled person can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest”. Unless otherwise specified, references to the numbering of specific amino acid residue positions in an antibody or antigen-binding portion thereof according to the invention are according to the Kabat numbering system.

Antibodies or antigen-binding portions thereof include, but are not limited to, polyclonal, monoclonal, multispecific, human, humanized, or chimeric antibodies, single-chain antibodies, epitope-binding fragments, e.g., Fab, Fab′ and F(ab′)₂, Fd, Fvs, single-chain Fvs (scFv), disulfide-linked Fvs (sdFv), fragments comprising either a VL or VH domain, or fragments produced by a Fab expression library. Immunoglobulin or antibody molecules of the disclosure can be of any class (e.g., IgG, IgE, IgM, IgD, IgA, and IgY) or subclass (e.g., IgG1, IgG2, IgG3, IgG4, IgA1, and IgA2, etc.) of immunoglobulin molecule.

The skilled person knows how to produce the antibodies according to the invention, e.g., in suitable cell culture systems.

All embodiments described herein in connection with an antibody of the invention are also meant to be disclosed for an antigen-binding fragment thereof.

In an embodiment of the invention, the isolated antibody or antigen-binding portion thereof for use in the in vitro method is a Fab fragment. The use of Fab fragments in the in vitro method of the invention has the advantage that such fragments can be easily produced in a cost-efficient manner via bacterial expression followed by purification rather than only by a costly expression in eukarotic expression systems. In one embodiment of the invention, the labeled anti-DLL1 detection antibody is a labeled monovalent Fab fragment. In preferred embodiment of the invention, the labeled anti-DLL1 detection antibody is a labeled bivalent Fab fragment. Such bivalent Fab fragments have still the advantage that they can be produced in cost-efficient way in a bacterial expression system. Moreover, experiments of the inventors have shown that while both the labeled monovalent and bivalent Fab fragments as labeled detection antibodies are suitable to detect DLL-1 both quantitatively and qualitatively, the labeled bivalent Fab fragments as labeled detection antibodies provide additional enhancement of the intensities of the measured signal, and thus an even more reliable and sensitive detection when used in a method or assay according to the invention.

The term “capture antibody” as used herein refers to an antibody or antigen-binding portion thereof that is adsorbed to a test surface. Such an adsorption of the capture antibody to a test surface can be achieved by incubating the test surface with the capture antibody so that the capture antibody is adhered to the test surface. “Adhered to” in this context is meant in the sense of “bound to” or “attached to” and thus represents a substantially permanent adhesion. The test surface can be any suitable surface. In case the in vitro method of the invention is designed as an ELISA, the test surface can be a plastic surface, preferably a polystyrene surface.

The term “labeled detection antibody” as used herein refers to an antibody or antigen-binding portion thereof that detection of DLL1 protein via its label. In a preferable embodiment, this labeled detection antibody enables qualitative and/or quantitative detection of DLL1 protein via its label.

The nature of the label which is used for the anti-DLL1 detection antibody depends on the employed format of the in vitro method according to the invention. In this regard, an embodiment of the invention is that the in vitro method is an enzyme-linked immunosorbent assay (ELISA) method. In this case, the label of the anti-DLL1 detection antibody is any label suitable as a detection label known by the skilled person. Preferably, the label of the anti-DLL1 detection antibody is selected from the group consisting of alkaline-phosphatase, horse-radish-peroxidase, biotin, streptavidin, a fluorescent tag and a radioactive isotope.

These labels and the biochemical principles underlying their detection are known to the skilled person. A radiolabeled detection antibody can, e.g., be detected by measuring the radioactivity with a radiometric detector. Fluorescent tags can be detected by measuring the emitted fluorescent light. The reporter enzymes alkaline-phosphatase and horse-radish-peroxidase are typically used to catalyze reactions which lead to a measurable colored product. A suitable colorimetric substrate for alkaline phosphatase is for example 4-nitrophenyl phosphate disodium salt hexahydrate (pNPP). Upon dephosphorylation of pNPP, a water soluble yellow product is obtained which has a strong absorption at 405 nm. Absorption at 405 nm can be measure with an ELISA reader. Other suitable colorimetric substrates for alkaline phosphatase are 5-bromo-4-chloro-3-indolyl phosphate (BCIP) and nitroblue tetrazolium (NBT). Suitable colorimetric substrate for horse-radish peroxidase are for example 3,3′,5,5′ tetramethylbenzidine (TMB) and 2,2′-azino-di [3-ethylbenzthiazoline]sulfonate (ABTS).

In another embodiment of the invention, the in vitro method is a lateral flow assay method. In this case, the label of the detection antibody is preferably selected from the group consisting of a gold particle, a colored cellulose particle and a colored latex particle. Upon accumulation of the labeled detection antibody on the test line of the lateral flow assay, the detection can be performed visually by inspecting the coloring of the test line and/or the intensity of the test line can be measured by a reader or scanner.

The antigen for the antibodies of the invention is DLL1 protein, specifically human DLL1 protein. The sequence of human DLL1 can have the sequence of SEQ ID NO.: 21 or 22. The term “DLL1 protein” as used herein comprises post-translational modifications, as well as natural proteolytic and processed DLL1 protein. It also comprises the soluble, insoluble DLL1 protein and naturally occurring isoforms of DLL1 protein of SEQ ID NOs: 21 or 22 (UniProtKB-000548 (DLL1_HUMAN).

The in vitro method of the invention is preferably based on option c, wherein a labeled anti-DLL1 detection antibody and an anti-DLL1 capture antibody according to alternative i) and/or ii) are used. The research underlying the invention surprisingly showed that the anti-DLL1 capture antibody and the labeled anti-DLL1 detection antibody of alternative i) and/or ii) work particularly well in combination with each other to specifically and/or preferentially bind and detect DLL1 protein at low levels.

In another embodiment of the invention, a labeled anti-DLL1 detection antibody and an anti-DLL1 capture antibody are employed, wherein the labeled anti-DLL1 detection antibody and the anti-DLL1 capture antibody do not belong the same alternative i) or ii). In this case, the qualitative and quantitative detection of DLL1 protein in the in vitro method of the invention is even more efficient and reliable.

In a particularly preferred embodiment of the invention, a labeled anti-DLL1 detection antibody and an anti-DLL1 capture antibody are employed, wherein the labeled anti-DLL1 detection antibody is selected from alternative i) and the anti-DLL1 capture antibody from alternative ii). Experiments of the inventors have shown that in this case particularly good results are achieved in terms of qualitative and quantitative detection of DLL1 protein. In this case, particularly low levels of DLL1 protein can be detected.

The in vitro method can further comprise the following steps:
a) adsorbing an anti-DLL1 capture antibody onto a test surface;
b) incubating the test-surface-bound anti-DLL1 capture antibody with a mixture comprising
   a sample to be analyzed for the presence of DLL1 protein and
   a labeled anti-DLL1 detection antibody;
c) forming a complex comprising the anti-DLL1 capture antibody, the DLL1 protein and the labeled anti-DLL1 detection antibody; and
d) detecting the complex-bound labeled anti-DLL1 detection antibody.

In step a) of the in vitro method of the invention, the anti-DLL1 capture antibody is adsorbed onto a test surface.

Optionally, after step a) and specifically in case the in vitro method of the invention has the format of an ELISA, free binding sites on the test surface can be blocked. This prevents unspecific binding of the labeled anti-DLL1 detection antibody. To this aim, suitable solutions, so-called blocking solutions, are known to the skilled person from other ELISA formats. A blocking solution always contains a blocking agent. The blocking agent can be a protein or a mixture of proteins. In particular, the blocking agent can be bovine serum albumin (BSA), newborn calf serum (NBCS), casein, non-fat dry milk or gelatin. Preferably, the blocking agent is BSA.

In step b) the test-surface-bound anti-DLL1 capture antibody is incubated with a mixture comprising
a sample to be analyzed for the presence of DLL1 protein and
a labeled anti-DLL1 detection antibody.

As a sample in this step of the in vitro method of the invention, typically a human sample is used, for which the DLL1 protein content needs to be assessed. Preferably, the sample is a human plasma sample or a human serum sample. Experiments of the inventors have shown that the antibodies of the invention are particularly suited to detect DLL1 protein in these kinds of samples.

If the sample contains DLL1 protein, the mixture typically contains complexes comprising DLL1 protein and labeled anti-DLL1 detection antibody.

The term “incubation” in the context of step b) means that the test-surface-bound anti-DLL1 capture antibody is exposed to the mixture comprising the sample and the labeled anti-DLL1 detection antibody. This exposure can be a “static exposure” or a “dynamic exposure” to the mixture. In case of the “static exposure” the test-surface bound anti-DLL1 capture antibody is continuously exposed to the mixture for a certain amount of time (e.g., in a well of an ELISA suitable well plate).

During the “dynamic exposure” the mixture passes by the test-surface bound capture antibody. In a preferred embodiment, the method step b) comprises a dynamic exposure incubation in a lateral flow assay, in particular a lateral flow immunoassay. In certain embodiments, the incubation period of step b) has a duration of 15 minutes or less, preferably 12 minutes or less. In a particularly preferred embodiment, the incubation of step b), and in particular dynamic exposure incubation in a lateral flow assay, has a duration of 10 minutes or less, 8 minutes or less, 5 minutes or less, 4 minutes or less, 3 minutes or less, 2 minutes or less, or 1 minute or less. The incubation step b) preferably has an incubation time of 10 minutes or less, or particularly preferred 5 minutes or less. This allows for a rapid diagnosis of a severe infection, in particular a sepsis.

In step c) a complex comprising the anti-DLL1 capture antibody, the DLL1 protein and the labeled anti-DLL1 detection antibody is formed. Due to the test-surface-binding of the anti-DLL1 capture antibody, this complex is also attached to the test surface.

In step d) the complex comprising the anti-DLL1 capture antibody, the DLL1 protein and the labeled anti-DLL1 detection antibody is detected qualitatively and quantitatively based on the label of the anti-DLL1 detection antibody.

The in vitro method of the invention can still comprise further steps, e.g., washing steps to remove any unbound material, in particular unbound labelled anti-DLL1 detection antibodies.

In a preferred embodiment, the in vitro method of the invention is performed, at least in part, in a lateral flow assay, in particular in a lateral flow immunoassay. This has the advantage that it allows qualitative and/or quantitative determination of DLL1 protein levels in the to-be-analyzed sample and that it can be performed at the time and place of patient care to diagnose a severe infection, in particular a sepsis. At the same time a minimum degree of skill and involvement from the user is required while at the same time a reliable result is obtained in a low cost format of diagnostic testing.

In a typical lateral flow assay, also known as a dipstick assay, the labeled detection antibody, i.e. here the labeled anti-DLL1 detection antibody as described above and in the examples below, is releasably immobilized in a conjugate pad. As soon as a sample is applied to the sample pad, capillary forces transport the sample via the conjugate pad, then the test line, optionally followed by the control line, to a wicking pad. If present in the sample, the labeled (anti-DLL1) detection antibody binds to target molecule (here the DLL1 protein). These complexes are then captured in the test line by the capture antibody (here the anti-DLL1 capture antibody). Preferably, the anti-DLL1 capture antibody binds to a different epitope on DLL1 as compared to the labeled anti-DLL1 detection antibody.

In one embodiment, the in vitro method of the invention further comprises a quantitative determination of DLL1 protein levels in the test line by
comparing the test line intensity to a reference, for example a reference card; or
by reading out the test line intensity with an optical measurement device.

Suitable optical measurement devices are known to the skilled person, e.g., an ESEQuant reader could be used.

Experiments of the inventors have shown that particularly good results are achieved in terms of qualitative and quantitative detection of DLL1 protein when the in vitro method of the invention is performed in a lateral flow assay.

### The Use of the Capture Antibody and the Labeled Detection Antibody

The invention also concerns the use of the labeled anti-DLL1 detection antibody and/or the anti-DLL1 capture antibody in an in vitro method to diagnose a severe infection, in particular a sepsis.

For the antibodies of the invention used in this way, the same embodiments as described above in connection with the in vitro method of the invention apply.

The term “severe infection” as used herein refers to a particularly evasive medical condition, which may result in life-threatening organ failure evoked by a dysregulated immune response to infection.

In certain embodiments, the labeled anti-DLL1 detection antibody and/or the anti-DLL1 capture antibody are used in an in vitro method to diagnose a severe infection, wherein the severe infection is selected from the group consisting of sepsis, pneumonia, and meningitis. In a preferred embodiment, the labeled anti-DLL1 detection antibody and/or the anti-DLL1 capture antibody are used in an in vitro method to diagnose a sepsis. In this regard, experiments of the inventors have shown that the antibodies of the invention are particularly suited to diagnose a sepsis since the antibodies of the invention are particularly suited to detect DLL1 protein in human blood samples, human plasma samples or human serum samples.

### Sequence Listing

This application contains a Sequence Listing which has been submitted electronically and is hereby incorporated by reference in its entirety. This sequence listing file is named 210071EP_Sequence Listing.txt and is 23.530 Bytes Bytes in size.

SEQ ID NO.: 1
(SYAMH) encodes for a CDR (VHCDR1) of AbD33905.
SEQ ID NO.: 2
(FISSGGSTTYYADSVKG) encodes for a CDR (VHCDR2)
of AbD33905.
SEQ ID NO.: 3
(FRSGYFDY) encodes for a CDR (VHCDR3) of
AbD33905.
SEQ ID NO.: 4
(RSSQSLVHSNGADYLN) encodes for a CDR (VLCDR1) of
AbD33905.
SEQ ID NO.: 5
(KGSNRDS) encodes for a CDR (VLCDR2) of AbD33905.
SEQ ID NO.: 6
(QQGYDFPPFT) encodes for a CDR (VLCDR3) of
AbD33905.
SEQ ID NO.: 7
(SYAIS) encodes for a CDR (VHCDR1) of AbD33906.
SEQ ID NO.: 8
(GIIPQFGIANYAQKFQG) encodes for a CDR (VHCDR2)
of AbD33906.
SEQ ID NO.: 9
(EGYSDYSAFDY) encodes for a CDR (VHCDR3) of
AbD33906.
SEQ ID NO.: 10
(TGTSSDVGTYNYVN) encodes for a CDR (VLCDR1) of
AbD33906.
SEQ ID NO.: 11
(RVSNRPS) encodes for a CDR (VLCDR2) of AbD33906.
SEQ ID NO.: 12
(SSWDEHSYV) encodes for a CDR (VLCDR3) of
AbD33906.
SEQ ID NO.: 13
(EVQLLESGGGLVQPGGSLRLSCAASGFTFSSYAMHWVRQAPGKGLEWV
SFISSGGSTTYYADSVKGRFTISRDNSKNTLYLQMNSLRAEDTAVYYCA
RFRSGYFDYWGQGTLVTVSS) encodesthe V_{H} domain of
AbD33905. The CDRs are underlined.
SEQ ID NO.: 14
(DIVMTQSPLSLPVTPGEPASISCRSSQSLVHSNGADYLNWYLQKPGQS
PQLLIYKGSNRDSGVPDRFSGSGSGTDFTLKISRVEAEDVGVYYCQQGY
DFPPFTFGQGTKVEIKRT) encodes the V_{L}domain of
AbD33905. The CDRs are underlined.
SEQ ID NO.: 15
(QVQLVQSGAEVKKPGSSVKVSCKASGGTFSSYAISWVRQAPGQGLEWM
GGIIPQFGIANYAQKFQGRVTITADESTSTAYMELSSLRSEDTAVYYCA
REGYSDYSAFDYWGQGTLVTVSS) encodesthe V_{H} domain of
AbD33906. The CDRs are underlined.
SEQ ID NO.: 16
(DIALTQPASVSGSPGQSITISCTGTSSDVGTYNYVNWYQQHPGKAPKV
MIYRVSNRPSGVSNRFSGSKSGNTASLTISGLQAEDEADYYCSSWDEHS
YVFGGGTKLTVLGQ) encodes the V_{L}domain of AbD33906.
The CDRs are underlined.
SEQ ID NO.: 17
(EVQLLESGGGLVQPGGSLRLSCAASGFTFSSYAMHWVRQAPGKGLEWV
SFISSGGSTTYYADSVKGRFTISRDNSKNTLYLQMNSLRAEDTAVYYCA
RFRSGYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKS) encodes the V_{H} and CH1
domain of AbD33905. The CDRs are underlined.
SEQ ID NO.: 18
(DIVMTQSPLSLPVTPGEPASISCRSSQSLVHSNGADYLNWYLQKPGQS
PQLLIYKGSNRDSGVPDRFSGSGSGTDFTLKISRVEAEDVGVYYCQQGY
DFPPFTFGQGTKVEIKRTVAAPSVFIFPPSDEQLKSGTASVVCLLNNFY
PREAKVQWKVDNALQSGNSQESVTEQDSKDSTYSLSSTLTLSKADYEKH
KVYACEVTHQGLSSPVTKSFNRGEA) encodes the V_{L} and C_{L}
domain of AbD33905.The CDRs are underlined.
SEQ ID NO.: 19
(QVQLVQSGAEVKKPGSSVKVSCKASGGTFSSYAISWVRQAPGQGLEWM
GGIIPQFGIANYAQKFQGRVTITADESTSTAYMELSSLRSEDTAVYYCA
REGYSDYSAFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKS) encodes the V_{H} and
CH1 domain ofAbD33906. The CDRs are underlined.
SEQ ID NO.: 20
(DIALTQPASVSGSPGQSITISCTGTSSDVGTYNYVNWYQQHPGKAPKV
MIYRVSNRPSGVSNRFSGSKSGNTASLTISGLQAEDEADYYCSSWDEHS
YVFGGGTKLTVLGQPKAAPSVTLFPPSSEELQANKATLVCLISDFYPGA
VTVAWKADSSPVKAGVETTTPSKQSNNKYAASSYLSLTPEQWKSHRSYS
CQVTHEGSTVEKTVAPTEA) encodes the V_{L} and C_{L} domain
of AbD33906. The CDRsare underlined.

### EXAMPLES

### Example 1: Sandwich ELISA Demonstrates High Binding Affinity of the Antibodies of the Invention

Polystyrene ELISA plates (384 well Nunc® Maxisorp™ MTP, black, flatbottom, PS, Thermo Scientific, 10395991) were coated with 1 μg/ml of the capture antibody AbD33905 (Fab fragment, monovalent) or AbD33906 (Fab fragment, monovalent) dissolved in PBS (NaCl: 137 mM, KCl: 2.7 mM, Na₂HPO₄: 10 mM, KH₂PO₄: 1.8 mM). As control coating a-Fd (Bio-Rad antibodies, STAR126) at 5 μg/ml in PBS was used. Per well a volume of 20 μl coating solution was used. The Polystyrene ELISA plates were then incubated overnight at 4° C. with a closed lid. After removal of the coating solution, five washes with PBST (PBS with 0.05% (v/v) Tween® 20, Merck Millipore, 817072) were performed. Subsequently, blocking of non-specific binding sites was performed with 100 μl per well of PBST containing 5% (w/v) BSA (Sigma-Aldrich, A7906-100). This blocking step was conducted at RT for 1-2 h. After removal of the blocking solution, five washes with PBST were performed. Thereafter, 2 μg/ml DLL1 antigen in PBST were added or PBST only (20 μl per well, triplicates were prepared). Incubation with the DLL1 antigen solution or PBST was performed at RT for 1 h. Next, the DLL1 antigen solution or PBST were removed and five washes with PBST were performed. Thereafter 20 μl/well of 2 μg/ml labeled (HRP conjugated) detection antibodyAbD33905 (Fab fragment, monovalent) or ABD33906 (Fab fragment, monovalent) in HISPEC assay dilutent (Bio-Rad Antibodies, BUF049) were added and incubated at RT for 1 h. After removal of this solution, ten washes with PBST were performed. Subsequently, 20 μl/well of QuantaBlu® fluorsence detection reagent (ThermoScientific, 15169) were added. For detection in the ELISA reader (Tecan Infinite M1000Pro Reader, Tecan Austria GmbH) excitation was performed at 320±25 nm and emission was detected at 430±35 nm.

Table 2 depicts the results of this ELISA assay. The values therein reflect the ratio of the emission values measured (arbitrary units) when using the DLL1 antigen solution in the assay versus the emission values measured (arbitrary units) when performing the assay without an antigen (emission value_{DLL1 antigen}/emission value_{PBST}).

TABLE 2
Sandwich ELISA results for AbD33905 and AbD33906

Detection antibody

| Capture antibody | AbD33905 | AbD33906 |
|---|---|---|
| Ab33905 | 8 | 66 |
| AbD33906 | 132 | 7 |
| a-Fd | 1 | 1 |

The results of Table 2 clearly indicate that AbD33905 and AbD33906 are capable of specifically and/or preferentially binding and detecting DLL1 protein in a sandwich ELISA assay, and particularly good results are obtained when using the combination of AbD33906 as capture antibody and AbD33905 as labeled detection antibody.

### Example 2: Sandwich ELISA Demonstrates High Binding Affinity of the Antibodies of the Invention

Polystyrene ELISA plates (384 well Nunc® Maxisorp™ MTP, black, flatbottom, PS, Thermo Scientific, 10395991) were coated with 1 μg/ml of the capture antibody AbD33906 (Fab fragment, monovalent) dissolved in PBS (NaCl: 137 mM, KCl: 2.7 mM, Na₂HPO₄: 10 mM, KH₂PO₄: 1.8 mM). Per well a volume of 20 μl coating solution was used. The Polystyrene ELISA plates were then incubated overnight at 4° C. with a closed lid. After removal of the coating solution, five washes with PBST (PBS with 0.05% (v/v) Tween® 20, Merck Millipore, 817072) were performed. Subsequently, blocking of non-specific binding sites was performed with 100 μl per well of PBST containing 5% (w/v) BSA (Sigma-Aldrich, A7906-100). This blocking step was conducted at RT for 1-2 h. After removal of the blocking solution, five washes with PBST were performed. Thereafter, serial dilutions of DLL1 antigen in PBST were added (20 μl per well, triplicates were prepared). Incubation with the serial dilutions of DLL1 antigen was performed at RT for 1 h. Next, the serial dilutions of DLL1 antigen were removed and five washes with PBST were performed. Thereafter 20 μl/well of 2 μg/ml labeled (HRP conjugated) detection antibody AbD33905 (Fab fragment, monovalent) in HISPEC assay dilutent (Bio-Rad Antibodies, BUF049) were added and incubated at RT for 1 h. After removal of this solution, ten washes with PBST were performed. Subsequently, 20 μl/well of QuantaBlu® fluorsence detection reagent (ThermoScientific, 15169) were added. For detection in the ELISA reader (Tecan Infinite M1000Pro Reader, Tecan Austria GmbH) excitation was performed at 320±25 nm and emission was detected at 430±35 nm.

As comparative sandwich ELISAs, the above-described assay was performed in parallel with 12 different capture and labeled detection antibodies. The following pairs of capture and labeled detection antibodies are shown as representative examples:

| Capture antibody | Labeled detection antibody |
|---|---|
| (Fab fragment, | (HRP conjugated, |
| monovalent) | Fab fragment, monovalent) |
| AbD33908.1 | AbD33902.1 |
| AbD33908.1 | AbD33905 |
| AbD33908.1 | AbD33906 |

Table 3 summarizes the results of the above-described ELISA assay:

TABLE 3
Sandwich ELISA results of Example 2

| | | | DLL1 protein in PBST (ng/mL) | | | | | | | | | |
|---|---|---|---|---|---|---|---|---|---|---|---|---|
| | | | 32000 | 16000 | 8000 | 4000 | 2000 | 1000 | 500 | 250 | 125 | 63 |
| Emission | Capture antibody: | Repl. 1 | 30108 | 32168 | 31677 | 30306 | 26808 | 22222 | 28637 | 29550 | 25435 | 17950 |
| data in | AbD33906 | Repl. 2 | 32482 | 33970 | 32151 | 32911 | 29998 | 25537 | 24353 | 24501 | 23021 | 17009 |
| arbitrary | Labeled detection | Repl. 3 | 31509 | 34286 | 33184 | 35388 | 30610 | 29441 | 20902 | 22627 | 19005 | 13386 |
| units | antibody: AbD33905 | Avg. | 31366 | 33475 | 32337 | 32868 | 29139 | 25733 | 24631 | 25559 | 22487 | 16115 |
| | | SD (%) | 4 | 3 | 2 | 8 | 7 | 14 | 16 | 14 | 14 | 15 |
| | Control 1 | Repl. 1 | 34241 | 29593 | 31086 | 31531 | 28422 | 22496 | 12375 | 12445 | 9349 | 4822 |
| | Capture antibody: | Repl. 2 | 32872 | 29861 | 29798 | 31747 | 29528 | 24125 | 10396 | 9457 | 7619 | 3725 |
| | AbD339801.1 | Repl. 3 | 32097 | 26923 | 27732 | 29988 | 29009 | 24320 | 10647 | 8829 | 6542 | 4373 |
| | Labeled detection | Avg. | 33070 | 28792 | 29539 | 31089 | 28986 | 23647 | 11139 | 10244 | 7837 | 4307 |
| | antibody: AbD33902.1 | SD (%) | 3 | 6 | 6 | 3 | 2 | 4 | 10 | 19 | 18 | 13 |
| | Control 2 | Repl. 1 | 30819 | 25279 | 25307 | 28727 | 28336 | 26005 | 12432 | 11558 | 8699 | 5232 |
| | Capture antibody: | Repl. 2 | 27213 | 22424 | 21172 | 24756 | 24789 | 22220 | 14373 | 11216 | 8258 | 5014 |
| | AbD339801.1 | Repl. 3 | 23369 | 21418 | 20303 | 22274 | 24846 | 21040 | 16061 | 12536 | 9341 | 6273 |
| | Labeled detection | Avg. | 27134 | 23040 | 22261 | 25252 | 25990 | 23088 | 14289 | 11770 | 8766 | 5506 |
| | antibody: AbD33905 | SD (%) | 14 | 9 | 12 | 13 | 8 | 11 | 13 | 6 | 6 | 12 |
| | Control 3 | Repl. 1 | 19131 | 17378 | 18204 | 17931 | 18128 | 14560 | 13700 | 9121 | 5888 | 3354 |
| | Capture antibody: | Repl. 2 | 16903 | 16652 | 17367 | 17254 | 16431 | 12571 | 12207 | 9281 | 5889 | 2707 |
| | AbD339801.1 | Repl. 3 | 17090 | 19399 | 17454 | 16452 | 17701 | 13293 | 13460 | 8975 | 5600 | 2855 |
| | Labeled detection | Avg. | 17708 | 17810 | 17675 | 17212 | 17420 | 13475 | 13122 | 9126 | 5792 | 2972 |
| | antibody: AbD33906 | SD (%) | 7 | 8 | 3 | 4 | 5 | 7 | 6 | 2 | 3 | 11 |

| | |
|---|---|
| Background mean | 174 |

| | | | DLL1 protein in PBST (ng/mL) | | | | | | | | | | | | |
|---|---|---|---|---|---|---|---|---|---|---|---|---|---|---|---|
| | | | 31 | 16 | 8 | 4 | 2.0 | 0.98 | 0.49 | 0.244 | 0.122 | 0.061 | 0.031 | 0.015 | 0.008 |
| Emission | Capture antibody: | Repl. 1 | 9714 | 4232 | 2179 | 2665 | 626 | 393 | 289 | 296 | 257 | 652 | 279 | 312 | 199 |
| data in | AbD33906 | Repl. 2 | 8057 | 3453 | 2593 | 1151 | 670 | 452 | 337 | 316 | 243 | 248 | 228 | 222 | 231 |
| arbitrary | Labeled detection | Repl. 3 | 7473 | 3351 | 2173 | 1340 | 695 | 441 | 336 | 259 | 239 | 276 | 242 | 234 | 238 |
| units | antibody: AbD33905 | Avg. | 8415 | 3679 | 2315 | 1719 | 664 | 429 | 321 | 290 | 246 | 392 | 250 | 256 | 223 |
| | | SD (%) | 14 | 13 | 10 | 48 | 5 | 7 | 9 | 10 | 4 | 58 | 11 | 19 | 9 |
| | Control 1 | Repl. 1 | 1813 | 921 | 1117 | 603 | 372 | 289 | 227 | 233 | 348 | 273 | 224 | 175 | 212 |
| | Capture antibody: | Repl. 2 | 1861 | 705 | 677 | 504 | 349 | 249 | 199 | 254 | 199 | 213 | 217 | 154 | 224 |
| | AbD339801.1 | Repl. 3 | 1647 | 659 | 750 | 435 | 288 | 226 | 175 | 141 | 208 | 254 | 246 | 163 | 299 |
| | Labeled detection | Avg. | 1774 | 762 | 848 | 514 | 336 | 255 | 200 | 209 | 252 | 247 | 229 | 164 | 245 |
| | antibody: AbD33902.1 | SD (%) | 6 | 18 | 28 | 16 | 13 | 13 | 13 | 29 | 33 | 12 | 7 | 6 | 19 |
| | Control 2 | Repl. 1 | 2692 | 1063 | 1159 | 645 | 387 | 287 | 248 | 160 | 130 | 143 | 119 | 135 | 202 |
| | Capture antibody: | Repl. 2 | 2208 | 960 | 463 | 267 | 166 | 121 | 108 | 90 | 189 | 111 | 119 | 383 | 125 |
| | AbD339801.1 | Repl. 3 | 2937 | 1264 | 496 | 339 | 203 | 143 | 129 | 99 | 137 | 130 | 124 | 92 | 96 |
| | Labeled detection | Avg. | 2612 | 1096 | 706 | 417 | 252 | 184 | 162 | 116 | 152 | 128 | 121 | 203 | 141 |
| | antibody: AbD33905 | SD (%) | 14 | 14 | 56 | 48 | 47 | 49 | 47 | 33 | 21 | 13 | 2 | 77 | 39 |
| | Control 3 | Repl. 1 | 1534 | 830 | 425 | 539 | 252 | 176 | 114 | 105 | 156 | 133 | 118 | 151 | 239 |
| | Capture antibody: | Repl. 2 | 1206 | 727 | 422 | 378 | 184 | 239 | 223 | 118 | 136 | 116 | 282 | 108 | 114 |
| | AbD339801.1 | Repl. 3 | 1583 | 739 | 550 | 262 | 183 | 323 | 268 | 199 | 195 | 126 | 141 | 157 | 129 |
| | Labeled detection | Avg. | 1441 | 765 | 466 | 393 | 206 | 246 | 202 | 141 | 162 | 125 | 180 | 139 | 161 |
| | antibody: AbD33906 | SD (%) | 14 | 7 | 16 | 35 | 19 | 30 | 39 | 36 | 18 | 7 | 49 | 19 | 42 |

| | |
|---|---|
| Background mean | 174 |

FIG. 1 depicts the results of table 3. The sandwich ELISA pair (AbD33906_{capture}/AbD33905_{Detection}) has by far the lowest EC₅₀ value. This sandwich ELISA pair showed a surprisingly low detection level, in fact the lowest detection level of all comparator pairs, as summarized in Table 4 below.

TABLE 4
Detection levels of Example 1's sandwich ELISA pairs

| | Limit of detection (ng/ml) |
|---|---|
| Capture antibody: AbD33906 | 0.2 |
| Labeled detection antibody: AbD33905 | |
| Control 1 | 1 |
| Capture antibody: AbD339801.1 | |
| Labeled detection antibody: AbD33902.1 | |
| Control 2 | 16 |
| Capture antibody: AbD339801.1 | |
| Labeled detection antibody: AbD33905 | |
| Control 3 | 18 |
| Capture antibody: AbD339801.1 | |
| Labeled detection antibody: AbD33906 | |

### Example 3: The Antibodies of the Invention Specifically Bind to DLL-1

The binding specificity of AbD33905 in form of a bivalent Fab fragment fused to alkaline phosphatase followed by a FLAG®-tag and Twin-Strep®-tag was tested in the following ELISA:

Polystyrene ELISA plates (384 well Nunc® Maxisorp™ MTP, black, flatbottom, PS, Thermo Scientific, 10395991) were coated with 5 μg/ml of antigens (BSA, N1-CD33-His6, GST or DLL-1) dissolved in PBS (NaCl: 137 mM, KCl: 2.7 mM, Na₂HPO₄: 10 mM, KH₂PO₄: 1.8 mM). Per well a volume of 20 μl coating solution was used. The Polystyrene ELISA plates were then incubated overnight at 4° C. with a closed lid. After removal of the coating solution, five washes with PBST (PBS with 0.05% (v/v) Tween® 20, Merck Millipore, 817072) were performed. Subsequently, blocking of non-specific binding sites was performed with 100 μl per well of PBST containing 5% (w/v) non-fat dry milk. This blocking step was conducted at RT for 1-2 h. After removal of the blocking solution, five washes with PBST were performed. Thereafter, AbD33905 in form of a bivalent Fab fragment fused to alkaline phosphatase followed by a FLAG®-tag and Twin-Strep®-tag in PBST was added (20 μl per well). Incubation with this AbD33905 Fab fragment was performed at RT for 1 h. Next, the AbD33905 Fab fragment containing solution was removed and five washes with PBST were performed. Thereafter 20 μl/well of 2 μg/ml anti-Strep-HRP secondary antibody (Bio-Rad antibodies, MCA2489P, 1:5000) in HISPEC assay diluent (Bio-Rad Antibodies, BUF049) were added and incubated at RT for 1 h. After removal of this solution, ten washes with PBST were performed. Subsequently, 20 μl/well of QuantaBlu® fluorsence detection reagent (ThermoScientific, 15169) were added. For detection in the ELISA reader (Tecan Infinite M1000Pro Reader, Tecan Austria GmbH) excitation was performed at 320±25 nm and emission was detected at 430±35 nm.

Table 5 summarizes the emission data obtained from the above-described ELISA.

TABLE 5
ELISA results of Example 3 for AbD33905 in form of
a bivalent Fab fragment fused to alkaline phosphatase
followed by a FLAG ®-tag and Twin-Strep ®-tag

| | | Emission data in arbitrary |
|---|---|---|
| | | units |
| Immobilized antigen | BSA | 71 |
| | N1-CD33-His6 | 46 |
| | GST | 70 |
| | DLL-1 | 15458 |

The results of Table 5 are depicted in FIG. 2. These data clearly show that the tested AbD33905 bivalent Fab fragment specifically binds DLL1 protein.

Furthermore, AbD33905 and AbD33906 do not recognize DLL3 and DLL4 (data not shown).

### Example 4: Lateral Flow Assay with Recombinant DLL-1 Protein

A lateral flow assay with recombinant DLL1 protein was performed with the following lateral flow device: The lateral flow test device contained a sample pad, a conjugate pad, a test line and a wicking pad. The sample pad contained a blood separator. The conjugate pad contained Fab fragments of AbD33905 coupled to gold particles in a concentration of 0.5 μg/cm. The test line contained Fab fragments of AbD33906 antibodies in a concentration of 0.5 μg/cm.

To the sample pad 100 μL of different recombinant DLL1 protein containing solutions were applied. The DLL1 protein containing solutions had concentrations of 0 ng/ml, 10 ng/ml, 25 ng/ml, 50 ng/ml, 100 ng/ml and 250 ng/ml. Each DLL1 solution was applied to two different lateral flow devices and analyzed after an assay time of 10 min. The intensity of the test lines was analyzed based on a gold standard reference card (GSK values from 1 to 10 with 1 corresponding to the lowest detection level and 10 corresponding to the highest detection level). The results of the lateral flow assays using recombinant DLL1 protein containing solutions are shown in FIG. 3. These results clearly indicate that recombinant DLL1 protein can be detected by the lateral flow assay in a concentration dependent manner starting from a DLL1 protein concentration of 10 ng/ml.

### Example 5: Lateral Flow Assay with Differently Conjugated AbD33905 Fab Fragments

To test whether other conjugates than gold particles are suitable for the lateral flow assay in combination with the antibodies of the invention, AbD33905 Fab fragments were coupled to Thiol-PEG-COOH gold particles (Thiol-PEG-COOH conjugate) or Latex (Nh2-Latex conjugate).

The following lateral flow device was used for this Example: The lateral flow test device contained a sample pad, a conjugate pad, a test line and a wicking pad. The sample pad contained a blood separator. The conjugate pad contained Fab fragments of AbD33905 coupled to gold particles, Thiol-PEG-COOH gold particles or Latex. The test line contained AbD33906 Fab fragments in a concentration of 0.5 μg/cm.

To the sample pad 30 μL of different recombinant DLL1 protein containing solutions were applied. The DLL1 protein containing solutions had concentrations of 0 ng/ml, 1 ng/ml, 10 ng/ml, 100 ng/ml, and 250 ng/ml. The lateral flow assays were analyzed after an assay time of 10 min.

The results of the lateral flow assays are depicted in FIG. 4, which clearly indicate that also differently conjugated AbD33905 Fab fragments are suitable for the lateral flow assay. The intensity of the test lines in FIG. 4 was analyzed based on a gold standard reference card (GSK values from 1 to 10 with 1 corresponding to the lowest detection level and 10 corresponding to the highest detection level).

### Example 6: Lateral Flow Assay with Human Blood Samples

The lateral flow assay with human plasma samples was performed with the following lateral flow device:

The lateral flow device contained a sample pad, a conjugate pad, a test line and a wicking pad. The sample pad contained a blood separator. The conjugate pad contained Fab fragments of AbD33905 coupled to gold particles in a concentration of 0.5 μg/cm. The test line contained AbD33906 Fab fragments in a concentration of 0.5 μg/cm.

To the sample pad 100 μL of different human plasma samples were added. While samples “Control 02”, “Control 10”, “Control 12”, “Control 16” and “Control 28” were from from healthy donors, samples “Sepsis Patient 01”, “Sepsis Patient 03”, “Sepsis Patient 06”, “Sepsis Patient 16” and “Sepsis Patient 30” were from patients suffering from a sepsis. The human samples were known to contain DLL1 protein in the amounts as indicated in Table 6 below. The lateral flow assays were analyzed after an assay time of 10 min.

TABLE 6
DLL1 protein concentration in tested human samples

| Sample | | DLL1 |
|---|---|---|
| Number | Sample name | [ng/ml] |
| 1 | Control 02 | 4.93 |
| 2 | Control 10 | 3.53 |
| 3 | Control 12 | 5.31 |
| 4 | Control 16 | 6.59 |
| 5 | Control 28 | 8.88 |
| 6 | Sepsis Patient 01 | 20.36 |
| 7 | Sepsis Patient 03 | 80.29 |
| 8 | Sepsis Patient 06 | 101.53 |
| 9 | Sepsis Patient 16 | 60.46 |
| 10 | Sepsis Patient 30 | 41.68 |

The results of the lateral flow tests are depicted in FIGS. 5 and 6. In FIG. 5, the intensity of the test lines was analyzed based on a gold standard reference card (GSK values from 1 to 10 with 1 corresponding to the lowest detection level and 10 corresponding to the highest detection level). FIG. 5 clearly shows that all lateral flow tests used for samples from patients suffering from a sepsis had GSK values above 3. The samples from healthy donors had GSK values of 0. Thus, the lateral flow assay is suitable to quantitatively detect DLL1 protein in human samples and thus to diagnose a sepsis.

The intensity of the test lines in the lateral flow devices can also be assessed with a reader, for example with an ESEQuant reader. FIG. 6 depicts the intensities of the test lines in the lateral flow tests of Example 6 together with a calibration curve obtained by using recombinant DLL1 protein solutions for the lateral flow tests. Again, this highlights that the lateral flow devices are suitable to detect DLL1 protein in human samples and thus to diagnose a sepsis. As evidenced by the increase in signal with increasing DLL1 protein concentration levels measured in the samples, this also evidences that the lateral assay of the present invention can be used, not only to detect, but also to qualitatively and/or quantitatively measure the level of DLL1 protein in biological samples.

### Example 7: Comparison of Monovalent AbD33905 to bivalentAbD33905 as Detection Fab Fragments

The lateral flow assay was performed with the following lateral flow device:

The lateral flow device contained a sample pad, a conjugate pad, a test line and a wicking pad. The sample pad contained a blood separator. The conjugate pad contained either monovalent Fab fragments of AbD33905 coupled to gold particles or bivalent Fab fragments of AbD33905 coupled to gold particles, in a concentration of 0.5 μg/cm. The test line contained AbD33906 Fab fragments in a concentration of 0.5 μg/cm.

The following samples were applied to the sample pad: Citrate blood samples were spiked with recombinant DLL1 protein to mimic human plasma samples with DLL1 protein concentrations according to Table 6 above.

Plasma was then also obtained from the citrate samples spiked with recombinant DLL1 protein. 100 μL of citrate blood samples spiked with recombinant DLL1 protein and having DLL1 concentrations according to Table 6 as well as plasma samples derived therefrom were applied to the sample pad. The lateral flow assays were analyzed after an assay time of 10 min using a gold standard reference card or an ESEquant reader.

The results of the lateral flow tests using the citrate blood samples are depicted in FIG. 7 and the ones of the lateral flow tests using the plasma samples derived from the citrate blood samples are depicted in FIG. 8. In FIGS. 7 and 8, the intensity of the test lines was analyzed based on a gold standard reference card (GSK values from 1 to 10 with 1 corresponding to the lowest detection level and 10 corresponding to the highest detection level). FIGS. 7 and 8 clearly show that the both the monovalent and bivalent AbD33905 labeled detection antibody is used, but that signal intensities in the test line are even higher and thus produce even better, more reliable, results when using the bivalent AbD33905 as labeled detection antibody.

The intensity of the test lines was also assessed with an ESEquant reader. The results for representative patient and control samples are shown in FIGS. 9 and 10. Again, higher signal intensities at the test line were measured when using the bivalent AbD33905 as labeled detection antibody, however both the monovalent and bivalent forms reliably and definitively was able to identify the sepsis patients, both with plasma and blood samples.

### BRIEF DESCRIPTION OF THE DRAWINGS

FIG. 1 depicts the sandwich ELISA results of Table 3 (see Example 2).

FIG. 2 depicts the ELISA results of Table 5 (see Example 3).

FIG. 3 depicts the lateral flow test results of Example 4 using recombinant DLL1 containing samples. The intensity of the test lines was assessed visually using a gold standard reference card.

FIG. 4 depicts the lateral flow test results of Example 5 using differently conjugated AbD33905 Fab fragments. The intensity of the test lines was assessed visually using a gold standard reference card.

FIG. 5 depicts the lateral flow test results of Example 6 using human DLL1 containing samples. The intensity of the test lines was assessed visually using a gold standard reference card.

FIG. 6 depicts the lateral flow test results of Example 6. The intensity of the test lines was determined with a reader.

FIG. 7 depicts the lateral flow test results of Example 7 using the citrate blood samples. The intensity of the test lines was assessed visually using a gold standard reference card.

FIG. 8 depicts the lateral flow test results of Example 7 using the plasma sample derived from the citrate blood samples. The intensity of the test lines was assessed visually using a gold standard reference card.

FIG. 9 depicts the lateral flow test results of the citrate blood samples and the plasma samples derived therefrom according to Example 7 using the bivalent AbD33905. The intensity of the test lines was determined with an ESEquant reader.

FIG. 10 depicts the lateral lateral flow test of the citrate blood samples and the plasma samples derived therefrom according to Example 7 using the monovalent AbD33905. The intensity of the test lines was determined with an ESEquant reader.

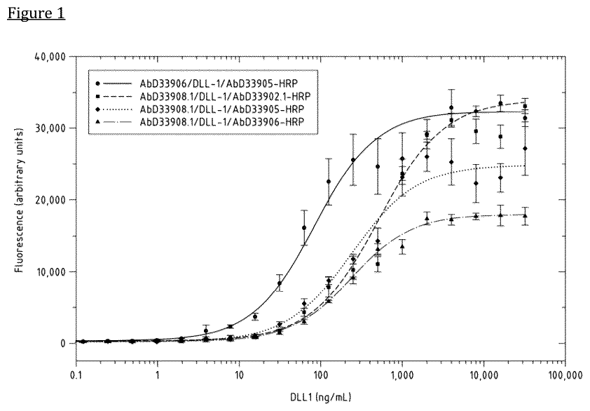

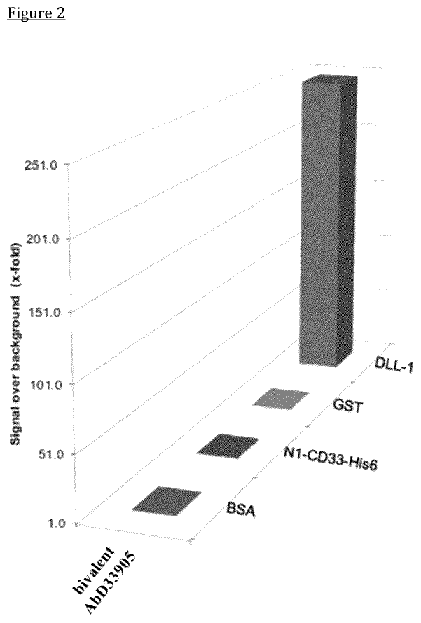

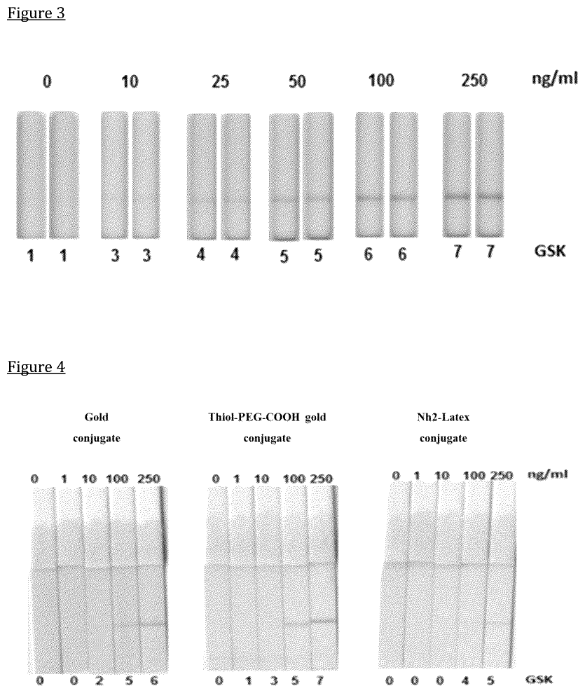

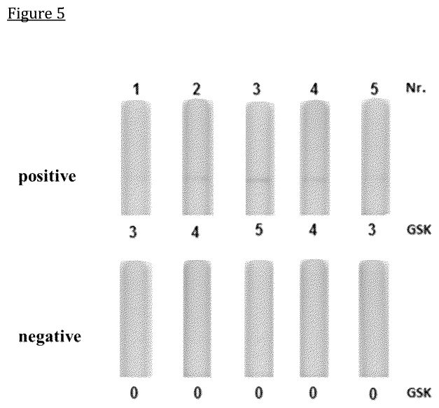

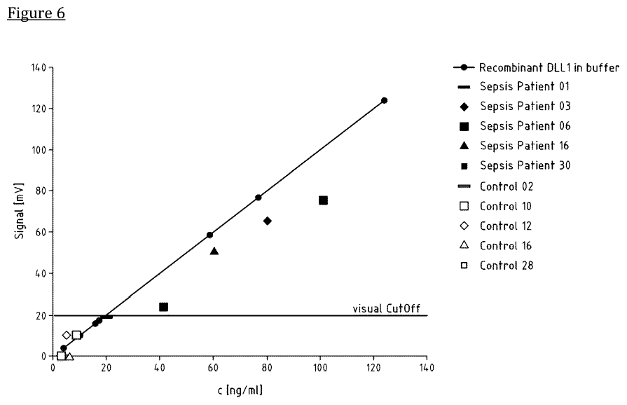

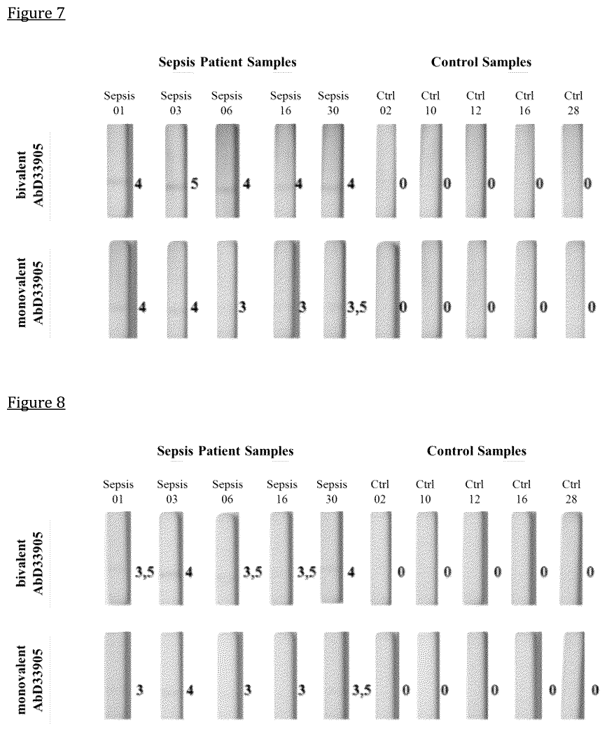

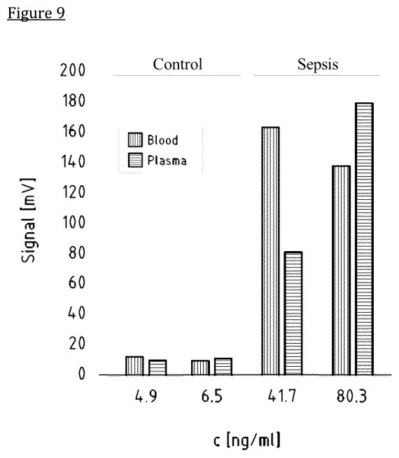

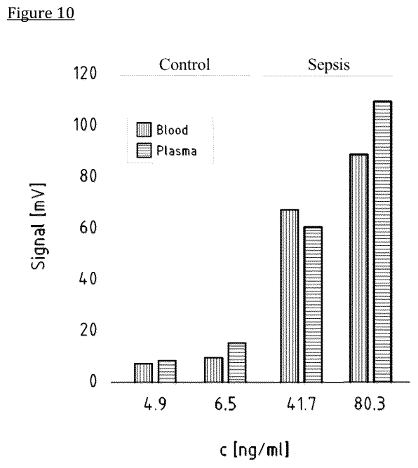

1. An in vitro method for detecting Delta-like 1 (DLL1) protein using
a. a labeled anti-DLL1 detection antibody, or
b. an anti-DLL1 capture antibody, or
c. a labeled anti-DLL1 detection antibody and an anti-DLL1 capture antibody
wherein the labeled anti-DLL1 detection antibody and/or the anti-DLL1 capture antibody are selected from the group consisting of:
i) an isolated antibody or antigen-binding portion thereof comprising at least one complementary determining region (CDR) amino acid sequence selected from SEQ ID NOs.: 1-6; and
ii) an isolated antibody or antigen-binding portion thereof comprising at least one CDR amino acid sequence selected from SEQ ID NOs.: 7-12.

2. The in vitro method according to claim 1, wherein
a. the labeled anti-DLL1 detection antibody, or
b. the anti-DLL1 capture antibody, or
c. the labeled anti-DLL1 detection antibody and the anti-DLL1 capture antibody are selected from the group consisting of:
i) an isolated antibody or antigen-binding portion thereof comprising SEQ ID NO.: 1-6 as CDR amino acid sequences; and
ii) an isolated antibody or antigen-binding portion thereof comprising SEQ ID NO.: 7-12 as amino acid sequences.

3. The in vitro method according to claim 1, wherein
a. the labeled anti-DLL1 detection antibody, or
b. the anti-DLL1 capture antibody, or
c. the labeled anti-DLL1 detection antibody and the anti-DLL1 capture antibody are selected from the group consisting of:
i) an isolated antibody or antigen-binding portion thereof comprising
a heavy chain variable domain (VH) that has at least 95%, preferably at least 98% sequence identity to SEQ ID NO.: 13, and
a light chain variable domain (VL) that has at least 95%, preferably at least 98% sequence identity to SEQ ID NO.: 14; and
ii) an isolated antibody or antigen-binding portion thereof comprising
a heavy chain variable domain (VH) that has at least 95%, preferably at least 98% sequence identity to SEQ ID NO.: 15, and
a light chain variable domain (VL) that has at least 95%, preferably at least 98% sequence identity to SEQ ID NO.: 16.

4. The in vitro method according to claim 1, wherein in case of alternative c. the labeled anti-DLL1 detection antibody and the anti-DLL1 capture antibody do not belong the same alternative i) or ii).

5. The in vitro method according to claim 1, wherein in case of alternative c. the labeled anti-DLL1 detection antibody is selected from alternative i) and the anti-DLL1 capture antibody from alternative ii).

6. The in vitro method according to claim 1, wherein the isolated antibody or antigen-binding portion thereof is a Fab fragment.

7. The in vitro method according to claim 1, wherein the method comprises the steps of
a) Adsorbing an anti-DLL1 capture antibody onto a test surface;
b) Incubating the test-surface-bound anti-DLL1 capture antibody with a mixture comprising
i. a sample to be analyzed for the presence of DLL1 protein and
ii. a labeled anti-DLL1 detection antibody;
c) Forming a complex comprising the anti-DLL1 capture antibody, the DLL1 protein and the labeled anti-DLL1 detection antibody; and
d) Detecting the complex-bound labeled anti-DLL1 detection antibody.

8. The in vitro method according to claim 1, wherein the method is an enzyme-linked immunosorbed assay (ELISA) method or a lateral flow assay method.

9. The in vitro method according to claim 8, wherein in case the method is an enzyme-linked immunosorbed assay the label of the anti-DLL1 detection antibody is selected from the group consisting of alkaline-phosphatase, horse-radish-peroxidase, biotin, streptavidin, a fluorescent tag and a radioactive isotope, and in case the method is a lateral flow assay method the label of the anti-DLL1 detection antibody is selected from the group consisting of a gold particle, a colored cellulose particle and a colored latex particle.

10. Use of a labeled anti-DLL1 detection antibody and/or of an anti-DLL1 capture antibody as defined in claim 1 in an in vitro method to diagnose a severe infection, in particular a sepsis.
